(19) United States
(12) Patent Application Publication
(10) Pub. No.: US 2004/0033240 A1
(43) Pub. Date: Feb. 19, 2004

(54) Immunological combinations for prophylaxis and therapy of helicobacter pylori infection

(76) Inventors: Bruno Guy, Lyon (FR); Jean Haensler, Pollionnay (FR)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/312,416

(22) PCT Filed: Jul. 4, 2001
(86) PCT No.: PCT/EP01/09031

(30) Foreign Application Priority Data
Jul. 5, 2000 (EP) 00420148.9

(51) Int. Cl. A61K 39/02

(52) U.S. Cl. 424/234.1

(57) ABSTRACT

The invention relates to multivalent compositions for preventing or treating Helicobacter infections. Multivalent Helicobacter component compositions useful in prophylaxis comprises at least two, preferably three components, that are selected from AlpA, catalase, urcase, 525 protease and 76K proteins. Multivalent compositions useful in therapy include in particular 76K+caatalase+525 protease, urease+76K+catalase+525 protease, AlpA+76K+catalase+525 protease, AlpA+76K and AlpA+catalase.

### FIELD OF THE INVENTION

This invention relates to the fields of medicine, immunology and vaccinology. In particular, the invention relates to novel antigenic compositions and their use in immunological compositions or vaccines for the treatment and prevention of infection by Helicobacter pylori. The invention relates to multivalent compositions for preventing or treating Helicobacter infections. Multivalent Helicobacter component compositions useful in prophylaxis comprise at least two, preferably three components, that are selected from AlpA, catalase, urease, 525 protease and 76K proteins. Multivalent compositions useful in therapy includes in particular 76K+catalase+525 protease, urease+76K+catalase+525 protease, AlpA+76K+catalase+525 protease, AlpA+76K and AlpA+catalase.

### BACKGROUND OF THE INVENTION

Helicobacter pylori (H. pylori) infection is associated with significant gastroduodenal disorders, including gastritis, ulcers and gastroesophageal cancer (P. Correa 1995 Am. J. Surg. Pathol. 19 (suppl. 1) s37-s43; B. J. Marshall et al. 1984 Lancet 1: 1311-1315; J. Parsonnet 1995 Aliment Pharmacol. Ther. 9 (Suppl 2) 45-51). Various H. pylori antigens have been tested in animal models for their ability to elicit a protective immunological response against infection, using a variety of formulations and various routes of administration.

Various H. pylori proteins have been characterized or isolated so far. Antigens of H. pylori described to date include urease, which is composed of two subunits A and B of 30 and 67 kDa respectively (Hu & Mobley, Infect. Immun. (1990) 58: 992; Dunn et al., J. Biol. Chem. (1990) 265: 9464; Evans et al., Microbial Pathogenesis (1991) 10: 15; Labigne. et al., J. Bact. (1989) 173: 1920); the vacuole cytotoxin of 87 kDa (VacA) (Cover & Blaser, J. Biol. Chem (1992) 267: 10570; Phadnis et al., Infect. Immun. (1994) 62: 1557; WO 93/18150); and immunodominant antigen of 128 kDa associated with the cytotoxin (CagA, also called TagA) (WO 93/18150; U.S. Pat. No. 5,403,924); heat shock proteins HspA and HspB of 13 and 58 kDa respectively (Suerbvaum et al., Mol. Microbiol. (1994) 14: 959; WO 93/18150; a catalase of 54 kDa (Hazell et al., J. Gen. Microbiol. (1991) 137 : 57; F. J. Radcliff et al. 1997 Infect. Immun. 65: 4668-4674); a fibrillar haemaglutinin (HpaA) of 20 kDa; a histidine-rich protein of 15 kDa (JHpn) (Gilbert et al., Infect. Immun. (1995) 63 : 2682); an outer membrane protein of 30 kDa (Bölin et al., J. Clin. Microbiol. (1995) 33: 381); a membrane-associated lipoprotein of 20 kDa (Kostrcynska et al., J. Bact. (1994) 176: 5938) as well as a family of porins HopA, HopB, HopC and HopD, of molecular weight between 48 and 67 kDa (Exner et al., Infect. Immun. (1995) 63: 1567).

Some of these proteins have already been proposed as potential vaccinal antigens. In particular, urease is recognized as being a potential vaccine (WO 94/9823; WO 95/3824; WO 95/22987; Michetti et al., Gastroenterology (1994) 107: 1002; B. Guy, et al, (1998) Vaccine 16: 850; Guy et al. (1999) Vaccine 17: 1130).

Most studies conducted to date have involved urease formulated with potent mucosal adjuvants such as Cholera Toxin (CT), the Heat Labile Toxin of E. coli (LT) or their mutant non-toxic derivatives, using mucosal routes (mainly the intragastric route) of administration. Helicobacter sonicates, whole cells or different purified antigens have been shown to induce significant levels of protection (based on urease activity and/or culture and/or histology), especially in murine models. Unfortunately, most of the approaches described to date have involved the use of large doses of poorly characterized antigens, administered mucosally in the presence of toxic adjuvants and thus do not lend themselves to development of vaccines for use in humans. Thus, a need exists for a safe, efficacious vaccine for use in the treatment and prevention of H. pylori infection.

### SUMMARY OF THE INVENTION

For prophylactic applications (i.e., to induce a protective immunological response to keep an individual from becoming infected with H. pylori), it has now been found that certain combinations of H. pylori antigens can reduce the variability of protection induced with single antigens by systemic route in mice. Moreover, some antigen combinations induce a further reduction in colonization compared to antigens alone, in particular urease, which was heretofore thought to be the prototype antigen for H. pylori vaccine formulations.

Therefore, the invention provides for a composition comprising at least a first and second immunogenic Helicobacter components in a combined amount effective to generate a protective anti-Helicobacter immune response upon administration to an animal at risk of a Helicobacter infection, wherein said at least first and second immunogenic Helicobacter components are independently selected from the group consisting of:

(a) the Helicobacter AlpA protein or a peptide from said Helicobacter AlpA protein, or a nucleic acid that encodes said Helicobacter AlpA protein or peptide;

(b) the Helicobacter catalase protein or a peptide from said Helicobacter catalase protein, or a nucleic acid that encodes said Helicobacter catalase protein or peptide;

(c) the Helicobacter 76K protein or a peptide from said Helicobacter 76K protein, or a nucleic acid that encodes said Helicobacter 76K protein or peptide;

(d) the Helicobacter 525 protease or a peptide from said Helicobacter 525 protease, or a nucleic acid that encodes said Helicobacter 525 protease or peptide ; and

(e) the Helicobacter urease or a peptide from said Helicobacter urease, or a nucleic acid that encodes said Helicobacter urease or peptide;

provided that said first and second immunogenic Helicobacter components are different from each other.

It has also been found that a bivalent composition comprising (i) either AlpA and catalase or a 76K protein or (ii) a 76K protein (GHPO 1516, related to Bab A adhesin family) and GHPO 525 (protease) provides an efficacious therapeutic vaccine (i.e., for treating established infection). The therapeutic combination of 76K and 525 could also be improved by the addition of catalase. A fourth component such as urease or AlpA may be also suitable.

Therefore, the invention also relates to a composition comprising, in a combined amount effective to generate a significant therapeutic anti-Helicobacter immune response upon administration to an animal having a Helicobacter infection:

(a) the Helicobacter 76K protein or a peptide from said Helicobacter 76K protein ; or a nucleic acid that encodes said Helicobacter 76K protein or peptide; or an antibody, or antigen binding fragment thereof, that binds to said Helicobacter 76K protein or peptide; and

(b) the Helicobacter 525 protease or a peptide from said Helicobacter 525 protease; or a nucleic acid that encodes said Helicobacter 525 protease or peptide; or an antibody, or antigen binding fragment thereof that binds to said Helicobacter 525 protease or peptide; and, optionally,

(c) the Helicobacter catalase or a peptide from said Helicobacter catalase; or a nucleic acid that encodes said Helicobacter catalase or peptide; or an antibody, or antigen binding fragment thereof that binds to said Helicobacter catalase or peptide.

The invention also relates to a composition comprising at least a first and second immunogenic Helicobacter component in a combined amount effective to generate a significant therapeutic anti-Helicobacter immune response upon administration to an animal having a Helicobacter infection, wherein:

(a) said at least first immunogenic Helicobacter component is the Helicobacter AlpA protein or a peptide from said Helicobacter AlpA protein; or a nucleic acid that encodes said Helicobacter AlpA protein or peptide; or an antibody, or antigen binding fragment thereof, that binds to said Helicobacter AlpA protein or peptide; and

(b) said at least second immunogenic Helicobacter component is (i) the Helicobacter 76K protein or a peptide from said Helicobacter 76K protein; or a nucleic acid that encodes said Helicobacter 76K protein or peptide; or an antibody, or antigen binding fragment thereof, that binds to said Helicobacter 76K protein or peptide or (ii) Helicobacter catalase or a peptide from said Helicobacter catalase; or a nucleic acid that encodes said Helicobacter catalase or peptide;, or an antibody, or antigen binding fragment thereof, that binds to said Helicobacter catalase or peptide.

AlpA is a H. pylori adhesin. The amino acid sequence of AlpA of an H. pylori strain and corresponding nucleotide sequence are described in WO 96/41880.

The H. pylori catalase and/or corresponding gene are described in a number of publications including Newell et al, in Basic and clinical aspects of H. pylori infection (1994) Gasbarrini/Pretolani Eds, Hazell et al, J. Gen. Microbiol. Inf. Dis. (1992) 11: 522, WO95/27506, WO95/33482, Odenbreit et al, J. Bact. (December 1996) 178 (23): 6960 and WO 98/06853.

For use in the present invention, the H. pylori outer membrane of 76 kDa (76K) may be any one of the proteins of the 76 kD family that is described in WO 98/43479 as well as their corresponding genes, some of the family members e.g. BabB, being also described in WO 97/12908 and WO 97/47646.

The amino acid sequence and corresponding nucleotide sequence of the GHPO 525 protease of an H. pylori strain is described in WO 98/43478.

For use in the present invention, the proteins referred to herein above may comprise the amino acid sequence as described in the literature or any other amino acid sequence that is an allelic form of that actually described. The proteins may be used as such or alternatively, immunogenic peptides thereof.

Since their corresponding genes are also known, it is straightforward to produce each of the proteins by recombinant DNA techniques.

In addition to subunit vaccination, DNA vaccination is also proposed. Accordingly, the immunogenic components of the compositions of the invention may be also constituted by nucleic acids e.g., DNA molecules, encoding any of the proteins or peptides mentioned above; the encoding sequence being placed under the control of appropriate promoter for expression in an animal e.g., a mammal, for example humans. The CMV early promoter is useful for expression in mammals.

### BRIEF DESCRIPTION OF THE DRAWINGS

The invention is further described with reference to the accompanying figures, in which:

FIG. 1 shows the preparation of multivalent DC-Chol formulations. Briefly, 1: DC-Chol liposomes (A) are turned into DC-Chol/OG mixed micelles (B) by addition of an excess of detergent (OG) ; 2: the protein antigens are mixed into the solution; and 3: the detergent is removed by ultracentritugation to restore the liposomes in the presence of the antigens which can incorporate into the vesicles (C).

FIGS. 2a through c show immune responses against recombinant urease formulated with DC-Chol (DCC/U), as evidenced by antibody response in serum, interferon gamma (IFNγ) production by spleen cells and interleukin-10(IL-10) production by spleen cells (C+=positive control; C−=negative control);

FIG. 3 shows Western blot analysis of serum responses against antigen combinations (UreA subunit is not visible on the figure);

FIGS. 4a and b show prophylactic efficacy of the different antigen cocktails, as demonstrated by urease activity in stomach (bar=mean urease activity) and quantitative culture of H. pylori from stomach homogenates (bar=median cfu value) (Ur=Urease; Alp=AlpA; Ca=Catalase; Bb=BabB ; Pr=protease 525);

FIG. 5 shows the mean prophylactic efficacy (bacterial load after challenge) of the different antigen cocktails (A=no antigen; B=urease; C=cocktails);

FIG. 6 shows prophylactic efficacy of some monovalent vs bivalent combinations;

FIGS. 7a and b show therapeutic efficacy of the different antigen cocktails, as demonstrated by urease activity in stomach (bar=mean urease activity) and quantitative culture of H. pylori from stomach homogenates (bar=median cfu value) ; and

FIG. 8 shows therapeutic efficacy of some monovalent vs bivalent combinations.

### DETAILED DESCRIPTION OF THE INVENTION

Many different H. pylori antigens have been examined for their ability to elicit a protective immunological response. Candidate antigens such as Urease, Catalase, and VacA were first identified by “classical” fractionation techniques. More recently, the genome of two different strains of H. pylori has been sequenced, providing a large selection (literally hundreds) of different potential antigens to be cloned and characterized in vitro and in vivo (J. F. Tomb, et at. 1997 Nature 388: 539-547; R. A. Alm et al 1999 Nature 397: 176). Identifying which of these antigens will have the desired activity is no small task. Also, some antigens may work better in combination with others, further increasing the complexity of this determination. Finally, the recombinant nature of these candidate antigens provides an additional challenge; purification of such expressed recombinant proteins often requires the use of denaturing buffers (guanidium or urea). Such denaturing buffers often persist in high concentrations in the final product. This makes it difficult to test combinations of antigens, as such antigens must be mixed to prepare the combination, and the antigens to be used in combination often exist in different physico-chemical states (i.e., some being denatured in urea or guanidium, some being “native” or renatured in PBS). The problem to be solved is to identify combinations of H. pylori antigens capable of eliciting protective and/or therapeutic immunological responses in animals or humans, and to determine how to formulate such combinations of antigens so as to retain their essential immunological activity.

Ideally, such antigen combinations should be formulated together with an adjuvant. An adjuvant is a substance that enhances the immunogenicity of an antigen. Adjuvants may act by retaining the antigen locally near the site of administration to produce a depot effect, facilitating a slow, sustained release of antigen to cells of the immune system. Adjuvants can also attract cells of the immune system and may attract immune cells to an antigen depot and stimulate such cells to elicit an immune response.

Adjuvants have been used for many years to improve the host immune response to antigens of interest in vaccines, especially subunit or component vaccines comprised of recombinant proteins. Intrinsic adjuvants, such as lipopolysaccharides, normally are components of the killed or attenuated bacteria used as vaccines. Extrinsic adjuvants are immunomodulators that are typically non-covalently linked to antigens and are formulated to enhance the host immune response. Aluminum hydroxide and aluminum phosphate (collectively commonly referred to as alum) are routinely used as adjuvants in human and veteranary vaccines. Currently, alum is the only adjuvant licensed for human use, although hundreds of experimental adjuvants such as cholera toxin B are being tested. However, adjuvants such as cholera toxin B have deficiencies. For instance, while cholera toxin B is not toxic in the sense of causing cholera, even the most remote chance of minor impurity makes such adjuvants of limited applicability.

Adjuvants to be used in vaccine formulations for prevention and treatment should provide a “balanced” Th1/Th2 response, a profile likely to be associated with protective responses against H. pylori. The Th1 arm (a “cellular” response) has been shown to be critical in response to H. pylori infection. The Th2 arm (an “antibody” response) is also thought to be important. Thus, ideally, an adjuvant capable of stimulating both arms of the immune system, together with the correct combination of antigens, administered in the route most suited to eliciting the desired response, are all expected to be important components of a safe, efficacious vaccine for prophylaxis and therapy of H. pylori infection. One such balanced Th1/Th2 adjuvant is DC-Chol (F. Brunel et al. 1999 Vaccine 17: 2192-2203).

For use in a composition according to the invention, a protein or a polypeptide according to the invention may be formulated in or with liposomes, preferably neutral or anionic lipsomes, microspheres, ISCOMS or virus-like particles (VLPs), so as to promote the targeting of the protein or polypeptide or to enhance the immune response. Persons skilled in the art obtain these compounds without any difficulty, for example see Liposomes: A Practical Approach, RRC New ED, IRL press (1990).

The administration of immunological combinations of the present invention may be made as a single dose or as a dose repeated once or several times after a certain period. The appropriate dosage varies according to various parameters, for example the individual treated (adult or child), the vaccinal antigen itself, the mode and frequency of administration, the presence or absence of adjuvant and if present, the type of adjuvant and the desired effect (e.g. protection or treatment), as can be determined by persons skilled in the art. In general, an antigen according to the invention may be administered in a quantity ranging from 10 μg to 500 mg, preferably from 1 mg to 200 mg. In particular, it is indicated that a parenteral dose should not exceed 1 mg, preferably 100 μg. Higher doses may be prescribed for e.g. oral use.

Independently of the formulation, the quantity of protein administered to man by the oral route is for example of the order of 1 to 10 mg per dose, and at least 3 doses are recommended at 4-week intervals.

Another method of immunizing host animals, wholly apart from the “conventional” immunization regimens described hereinabove, concerns the use of “naked” DNA. Cells can be transfected with plasmid DNA containing gene sequences designed to express antigens of interest in transfected cells. Such transfection leads to transient expression of the exogenous DNA sequences, which can in turn induce humoral and/or cell mediated immunity. See, e.g., Felgner, et al (1994). J. Biol. Chem. 269, 2550-61. See also McClements et al., immunization with DNA vaccines encoding glycoprotein D or glycoprotein B, alone or in combination, induces protective immunity in animal models of herpes simplex virus-2 disease, PNAS USA 93:11414-11420, and U.S. Pat. Nos. 5,591,639, 5,589,466 and 5,580,859, the teachings of which are hereby incorporated herein in their entirety by reference. Thus, according to the present invention, combinations of H. pylori antigens, or immunological fragments thereof, or peptides containing protective epitopes, or nucleic acids encoding any of the aforementioned antigens, may be used to elicit a desired immunological response. In the alternative, any of the aforementioned antigens or nucleic acids can be administered to an animal, using conventional techniques, to raise antibodies or antibody fragments capable of binding to the H. pylori antigens of interest (e.g., urease, catalase, 76K, 525 and/or AlpA); such antibodies or antibody fragments can then be administered to a human or animal to passively protect against infection.

The invention is further illustrated by the following examples, which are meant to be illustrations only and are not intended to limit the present invention to specific embodiments.

### EXAMPLES

The practice of the present invention will employ, unless otherwise indicated, conventional methods of immunology, molecular biology, cell biology and recombinant DNA techniques known to those skilled in the art. Such techniques are explained fully in the literature. See, e.g., Sambrook, et al., Molecular cloning : A laboratory manual, Second Ed. (1989); Nucleic Acid Hybridization (B. D. Hames & S. J. Higgins, 1984); Animal Cell Culture (R Freshney ed. 1986); Immunochemical Methods in Cell and Molecular Biology (Mayer & Walker, eds., Academic press, London, 1987); Protein Purification: Principles and Practice, Second Edition (Scopes, ed., Springer-Verlag, N.Y. 1987); Current Protocols in Immunology (John Wiley & Sons, NY 1998); Antibodies, a Laboratory Manual (Ed Harlow and David Lane, eds, Cold Spring Harbor, N.Y. 1988); and Fundamental Immunology (Paul, ed, Raven Press, NY 1993).

Antigens and Adjuvants

Recombinant H. pylori urease was expressed and purified as previously described (Lee et al., J. Infect. Dis. (1995) 172: 161). Briefly, after cloning of the ureA and ureB genes under an inducible promoter and transformation in E. coli, inactive recombinant urease was expressed and purified from cell pellets. After several steps including ion-exchange chromatography and gel filtration, purified urease was lyophilized and stored at −20° C. After reconstitution, urease was stored at 4° C. The same urease preparation was used for all the experiments described in this study. It is usually resuspended in 20 mM Hepes buffer, pH 7.3 (=Hepes) to a final concentration of 4.0 mg/ml. This solution will also contain 2% sucrose from the lyophilisation ballast.

The other antigens were cloned in and expressed from E. coli. rAlpA, rCatalase, rBabB and rProtease are purified in denaturing buffers containing a chaotropic agent (e.g. urea, guanidinium, arginine).

Heat labile toxin from E. coil (LT) was purchased from Sigma (St Louis, USA).

Formulation of H. pylori Antigens

The DC-chol liposomal formulations are prepared by using the general detergent dialysis technique as described for instance by Weder, H. G. and Zumbuehi, O. The preparation of variably sized homogeneous liposomes for laboratory, clinical, and industrial use by controlled detergent dialysis. In Liposome technology: Gregoriadis G. (Ed.), Volume 1, CRC Press, Boca Raton, Fla. 1984; 79-105.

Briefly, chloroform solutions of lipids in the presence or absence of lipoidal adjuvants are mixed, evaporated, vacuum dessicated and resuspended in a buffer to yield a liposome suspension. This suspension is homogeneized by either extrusion, microfluidization or sonication and the resulting vesicles are then turned into lipid/detergent mixed micelles by the addition of excess detergent (e.g. alkyiglycosides, bile salt, etc.). The antigens of interest are then added to the mixed micelles to form an homogeneous solution. Finally the detergent is removed by controlled dialysis to restore the liposomes in the presence of the antigens.

Bacterial Challenge Strain

H. pylori X43-2AN, is a streptomycin resistant strain adapted to mice by serial passage (H. Kleanthous et al. VIIIth Int. Workshop on Gastroduodenal Path. and H. pylori. Jul. 7-9th, 1995, Edinburgh, Scotland, U.K.). This strain was stored at −70° C. in Brucella Broth (BB) (Biomerieux) supplemented with 20% v/v glycerol and 10% v/v foetal bovine serum (FBS) (Hyclone).

The challenge suspension was prepared as follows: for pre-culture, H. pyrlori was grown on Mueller-Hinton Agar (MHA; Difco) containing 5% v/v sheep blood (Biomerieux) and antibiotics: 5 pg.ml-1 Thrimethoprim, 10 pg.ml-1 Vancomycin, 1.3 pg.ml-1 Polymixin B sulfate, 5 pg.ml-1 Amphotericin and 50 μg.ml-1 Streptomycin (selective marker of strain X43-2AN) (TVPAS). All antibiotics were purchased from Sigma. MHA-TVPAS plates were incubated for 3 days at 37° C. under micro-aerobic conditions (Anaerocult C, Merck). The pre-culture was used to inoculate a 75 cm2 vented flask (Costar) containing 50 ml of BB supplemented with 5% v/v FBS and all antibiotics (TVPAS). The flask was kept under micro-aerobic conditions with gentle shaking for 24 hrs. The suspension was characterized by Gram's staining, urease activity (Urea indole medium, Diagnostic Pasteur), catalase (H202, 3% v/v) and oxidase activity (Biomerieux discs). Viability and motility were checked by contrast phase microscopy. The suspension was diluted in BB to OD 550 nm=0.1 (which was equivalent to 1×107 CFU.ml-1).

Animal Model of Infection

Outbred OF1 female mice 6-8-weeks-old were purchased from IFFA Credo (France). During the studies cages were covered (using Isocaps), mice were given filtered water and irradiated food and autoclaved material was used.

Mice were immunized on days 0, 21 and 42. Immunization was performed by the subcutaneous (SC) route (300 μl under the skin of the left part of the lumbar region). Five μg of recombinant H. pylori urease and of each antigen (alone or within the cocktails) were administered by sub-cutaneous (SC) route.

Mice were challenged 4 weeks after the second boost by gastric gavage with 300 μl of a suspension of H. pylori bacteria (3×10⁶ cfu).

In the therapeutic experiments, 10% of the infected mice (randomly selected) were analyzed by urease test one month after challenge. All mice were positive and the remaining animals were then immunized as previously described. Analysis of the challenge was done one month after the last immunization.

Evaluation of the Infection Rate

Four weeks after the challenge, mice were killed and stomachs were sampled to evaluate urease activity (Jatrox test, Procter and Gamble) in a sterile flow hood, and to perform culture and histological analyses. One half of the whole stomach (antrum+corpus) was taken for culture and/or one quarter for urease activity and histology according to the experiments. Urease activity was assessed 4 and 24 hrs postmortem by measuring the absorbance at 550 nm. The principle of the test is that the urea present in the test medium is split by H. pylori urease. The rise in pH causes a color change in the indicator which is likewise present in the test medium (phenol red)—from yellow to pink red.

Evaluation f Infection by Quantitative Culture

At post-mortem, the mucosa from one half stomach of each mouse was stored in the culture transport medium (Portagerm, Biomerieux) and transferred to the culture room within 2 hrs. The specimen was removed and homogenized with a sterile Dounce tissue grinder (Wheaton, Millville, USA) containing 1 ml of BB, and serial diluted to 10-3. One hundred μl of each dilution were inoculated onto MHA+TVPAS plates and incubated under micro-aerobic conditions at 37° C. for 4 to 5 days. Viable counts were recorded. H. pylori was identified by positive urease, catalase, oxidase and by typical appearance on Gram's stain.

Histology

A quarter of the stomach was placed in 10% buffered formalin (Labo-Modeme) and then processed for tissue sectioning. Sections were stained with hematoxylin and eosin (HE staining), and gastritis was scored based upon the infiltration of lymphocytes, plasma cells and neutrophils (Lee et al, 1995). Scoring was defined as follows: 0 no abnormalities; 1—a few leukocytes scattered in the deep mucosa; 2—moderate numbers of leukocytes in the deep to mid-mucosa, occasional neutrophils in glands; 3—dense infiltrates of leukocytes in the deep to mid mucosa, a few microabcesses, and 1 or 2 lymphoid aggregates; 4—dense, diffuse infiltrates of leukocytes throughout the lamina propria and into the submucosa, with prominent lymphoid aggregates, and several microabscesses filled with neutrophils.

Western Blot Analysis

Inactivated H. pylori bacteria were sonicated and total extract loaded on a SDS gel. After transfer of proteins and saturation with milk, the membrane strips were incubated with the different sera, and the presence of specific IgG1 and IgG2a antibodies detected according to standard procedures. Revelation was carried out with the ECL technique (Amersham).

Measurement of Cytokines/ELISPOTs with Spleen Cells

Nitrocellulose plates (Millipore) were coated with 5 μg/ml of anti mouse IL10 or IFNγ (Pharmingen). The spleens were teased through a 70 μm filter falcon). After treatment with Gey's solution to eliminate red cells and three further washes, the cells were counted and loaded into the wells of the plates at a final concentration of 2.10⁵ cells in 100 μl in each well. Three different concentrations (final concentration of 30,10 and 3 μg/ml) of filtrered H. pylori extract (containing 25% Urease) was added into the wells to stimulate the cells for 44 hours at 37° C. with 5% CO2. Each assay was done in triplicate in RPMI 1640 (Gibco) supplemented with 5% decomplemented FCS, sodium pyruvate, βME, glutamnine and antibiotics. A positive control (ConA, Sigma, at a 5 μg/ml final concentration) and a negative control (medium alone) were performed for each mouse. Secondary biotinylated anti mouse IL5 or γIFN antibodies (Pharmingen) were used at 1 μg/ml. Spots were revealed with AEC substrate (Sigma) and once the plates dried, counted with an automated spot counter (Microvision, France). The number of spots for 10⁶ cells induced by 10 μg/ml H. pylori extract was determined and the background (spots induced by medium alone, negative control) was substracted.

ELISAs

ELISAs were performed according to standard protocols (biotinylated conjugates, streptavidine peroxidase complex were from Amersham and OPD substrate from Sigma). Plates (Maxisorb, Nunc) were coated overnight at 4° C. with H. pylori extracts (5 ˜tg/ml) in carbonate buffer. After saturation with bovine serum albumin (Sigma), plates were incubated with the sera (1.5 hrs), biotinylated conjugate (1.5 hrs), streptavidin peroxidase complex (1 h) and substrate (10′). A polyclonal mouse serum directed against H. pylori extract served as a control in each experiment. The titers were expressed as the inverse of the dilution giving 50% of the maximal absorbance value at 492 nm.

Statistical Analysis

Protection was assessed by quantitative culture from infected stomachs and differences between groups was estimated by Newman Keuls and Dunetts tests.

### Example 1

Immune Responses Against H. pylori Urease

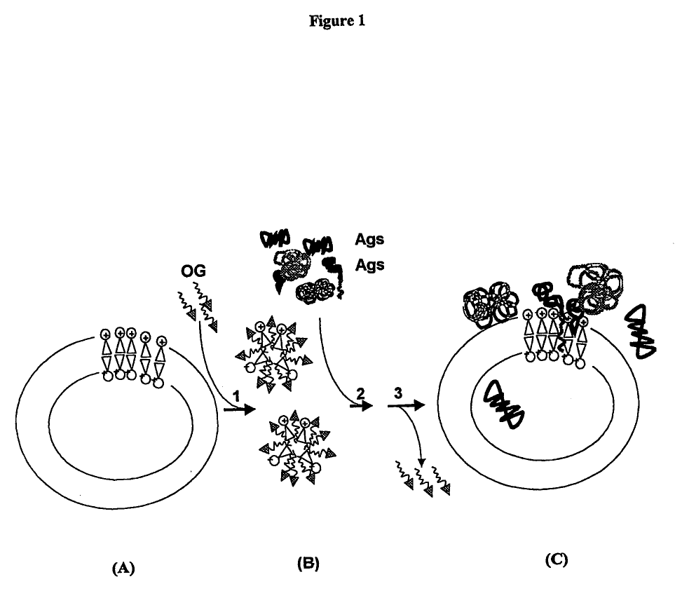

Urease administered with DC-Chol induced a balanced IgG1/IgG2a response in serum, and a predominant IFNγ response in spleen cells re-stimulated with urease invitro (FIG. 1). In similar experiments, urease administered with alum did not induce significant IFNγ production (not shown). Experiments carried out with six different preparations of urease/DC-Chol induced consistently the same pattern of immune responses in mice. This formed the base line to which efficacy of antigen combinations was compared.

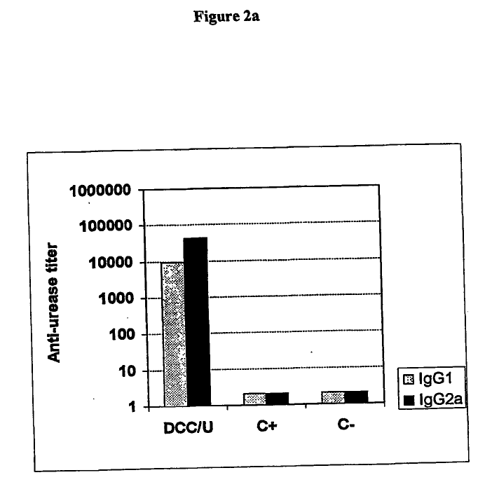

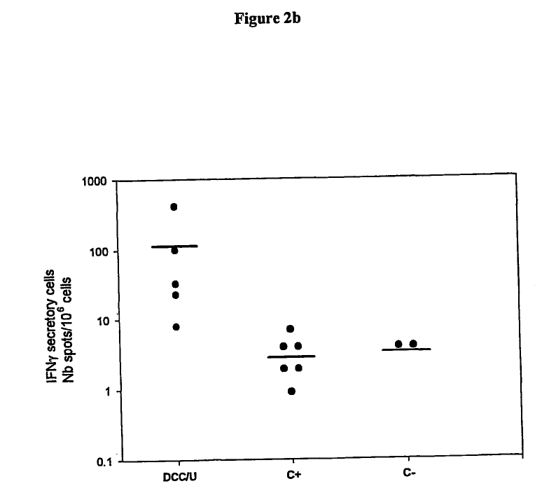

Specifically antigen combinations formulated in the balanced adjuvant were administered via the systemic route to compare the urease-induced protection to the one induced by the other antigens, alone or in combination Immune responses induced after immunization was examined by Western Blot. The results are shown in FIG. 2. For each formulation the expected reactivity was observed. Recombinant Urease and Catalase induced both IgG1 and IgG2a while AlpA, 76K and 525 induced a predominant IgG1 response. Recombinant 76K induced a reactivity against two different proteins or isoforms in the extract.

### Example 2

Antigen Combination for Prophylactic Immunization

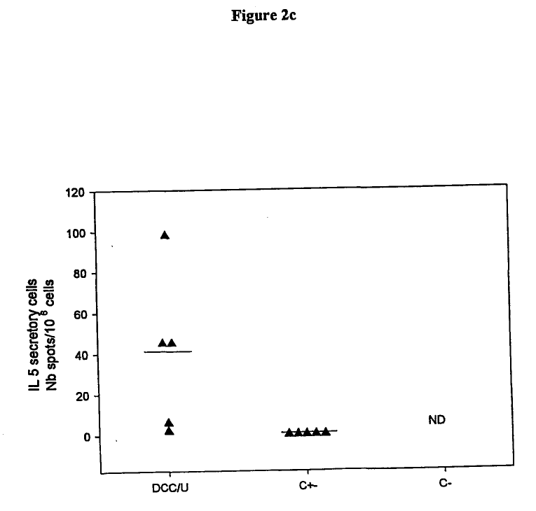

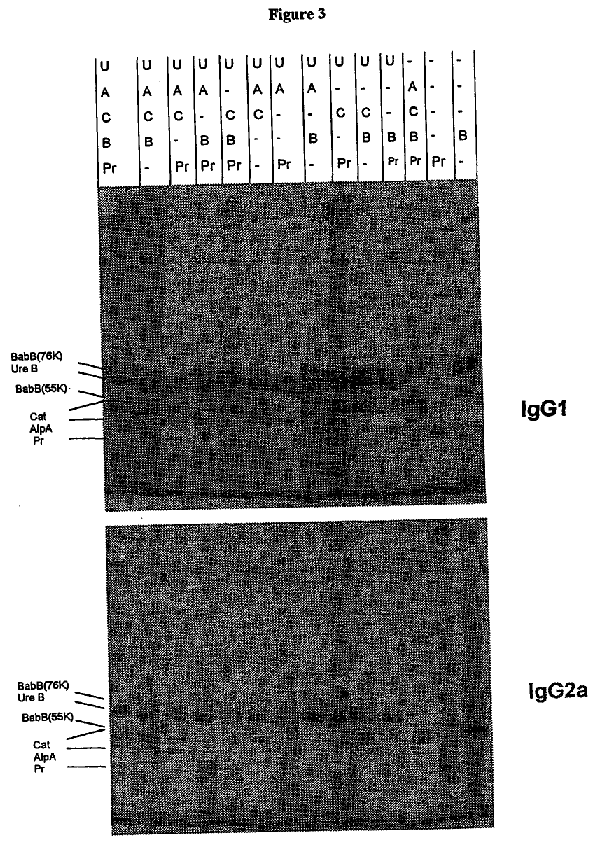

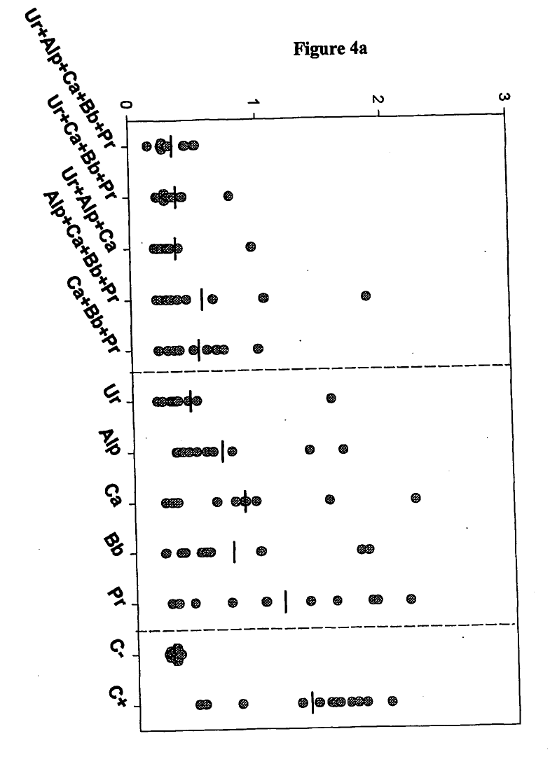

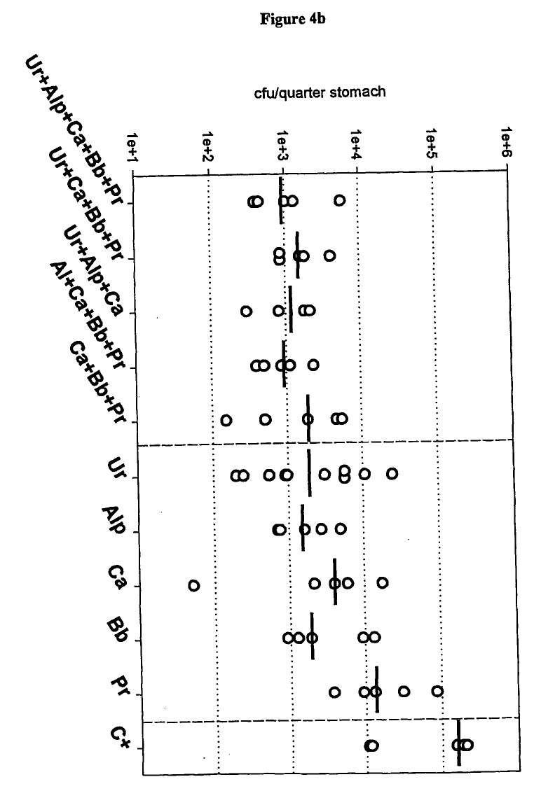

Antigen combinations were formulated and examined for their ability to induce protection in the animal model of infection described herein Protection was assessed by measuring the level of urease activity in the stomachs of all mice, and by quantitative culture in the stomach of all or half of the mice per group. The results are shown in FIG. 3. DC-Chol/urease induced a 2 log reduction in bacterial colonization (median cfu values), but in an heterogeneous way. Similar protection was achieved by the other antigens, except for protein 525 (p<0.05 except for this latter antigen compared to C+). The different cocktails also induced a 2-log reduction in bacterial density, but this was more homogeneous for most of the combinations. While in antigens-alone groups, about 50% of mice had levels of bacteria higher than 3000 cfus (in particular in the urease group), less than 20% of the cocktail-groups presented such high values. Similarly, while less than 25% of the mice presented low bacterial counts (below 1000 cfus) in the antigen-only groups, more than 50% of the mice had such low values in the cocktail-groups. Similar more-homogeneous protection was observed using such cocktails in two other and separate experiments using DC-Chol or a combination of DC-Chol and Bay adjuvants. This combination provided a 3-log decrease in median cfu values, an unexpected synergistic result (p<0.05 compared to antigen alone).

### Example 3

Antigen Combination for Therapeutic Immunization

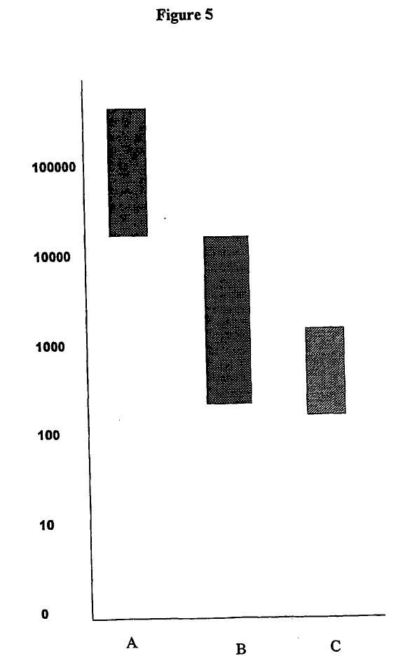

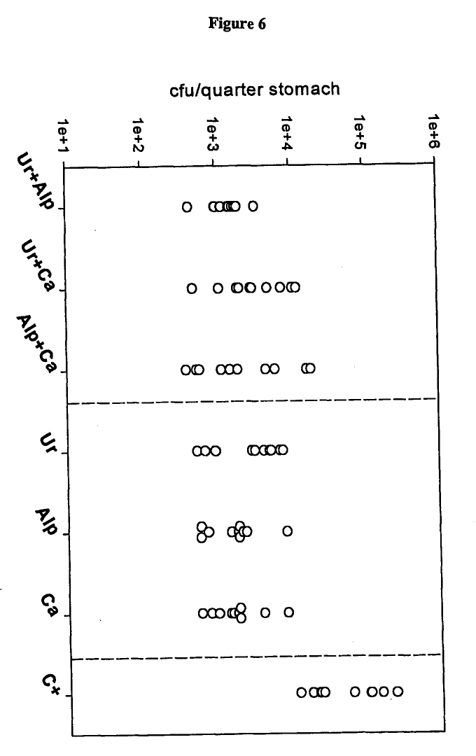

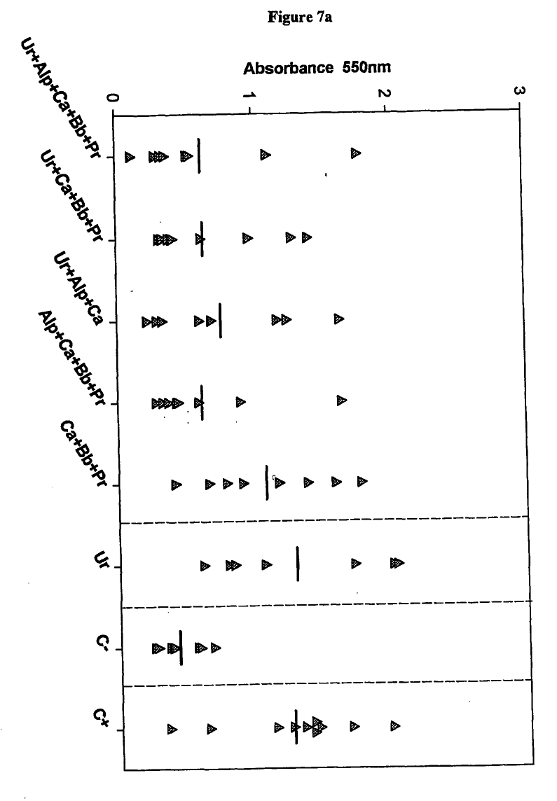

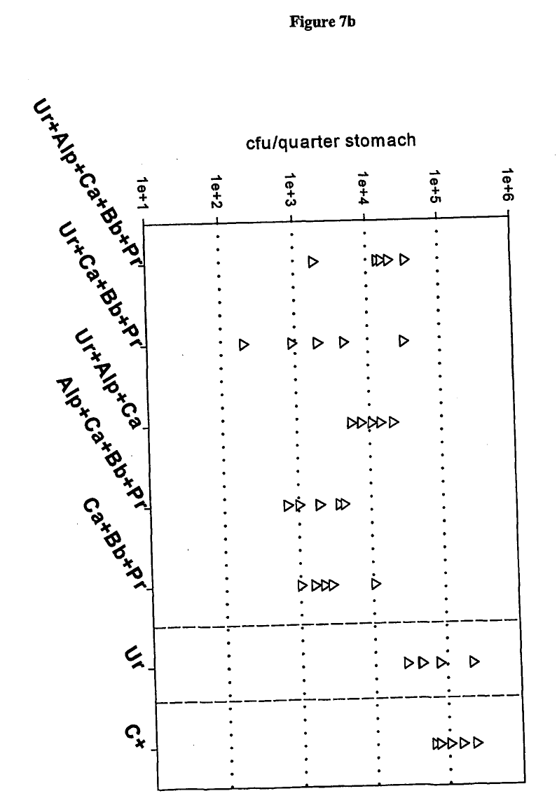

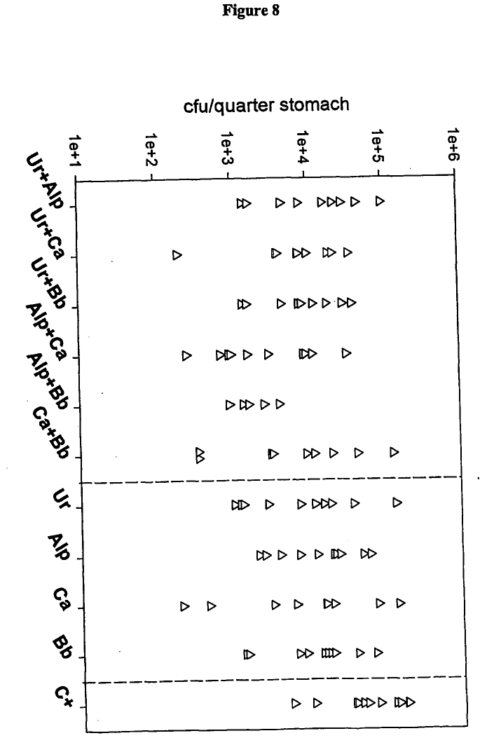

Therapeutic activity was assessed using the same formulations as described for Examples 1 and 2. Western blot analysis performed in mice immunized after challenge showed similar profiles than in the previous prophylactic experiment, indicating that prior colonization did not influence the level and the quality of the immune responses induced by the different formulations by systemic route. The different cocktails were then compared to urease in their ability to reduce colonization. As shown in FIG. 5, Urease formulated with DC-Chol did not induce a significant reduction in bacterial density, while some cocktails did. The cocktails containing Catalase, 76K and 525 induced the best levels of reduction (2 log in median cfu values, p<0.05). Contrarily to what we generally observed in prophylactic studies, where some correlation exists between urease activity and quantitative culture (the latter test being more sensitive), such a correlation was not really found among different therapeutic studies conducted in our lab, including the one presented here.

The level of gastritis was analyzed in some groups, that showed or not a reduced colonization. Although a moderate gastritis was observed in infected mice compared to uninfected mice (average score 1-2 in the former group vs 0-1 in the latter), no differences were observed in immunized-infected mice compared to unimmunized-infected mice (not shown), in agreement with previous studies.

1. A composition comprising at least a first and second immunogenic Helicobacter components in a combined amount effective to generate a protective anti-Helicobacter immune response upon administration to an animal at risk of a Helicobacter infection, wherein said at least first and second immunogenic Helicobacter components are independently selected from the group consisting of:
a) the Helicobacter AlpA protein or a peptide from said Helicobacter AlpA protein, or a nucleic acid that encodes said Helicobacter AlpA protein or peptide;
b) the Helicobacter catalase protein or a peptide from said Helicobacter catalase protein, or a nucleic acid that encodes said Helicobacter catalase protein or peptide;
c) the Helicobacter 76K protein or a peptide from said Helicobacter 76K protein, or a nucleic acid that encodes said Helicobacter 76K protein or peptide;
d) the Helicobacter 525 protease or a peptide from said Helicobacter 525 protease, or a nucleic acid that encodes said Helicobacter 525 protease or peptide; and
e) the Helicobacter urease or a peptide from said Helicobacter urease, or a nucleic acid that encodes said Helicobacter urease or peptide;
provided that said first and second immunogenic Helicobacter components are different from each other.

2. The composition according to claim 1, further comprising a third immunogenic Helicobacter component which is independently selected from the group consisting of (a), (b), (c), (d) and (e) as defined in claim 1; provided that said third immunogenic Helicobacter component is different from said first and second immunogenic Helicobacter components.

3. The composition according to claim 2, further comprising a fourth immunogenic Helicobacter component which is independently selected from the group consisting of (a), (b), (c), (d) and (e) as defined in claim 1; provided that said fourth immunogenic Helicobacter component is different from said first, second and third immunogenic Helicobacter components.

4. The composition according to claim 3, further comprising a fifth immunogenic Helicobacter component which is independently selected from the group consisting of (a), (b), (c), (d) and (e) as defined in claim 1; provided that said fifth immunogenic Helicobacter component is different from said first, second, third and fourth immunogenic Helicobacter components.

5. The composition according to any one of claims 1 to 4, wherein the 76K protein is BabB.

6. The composition according to any one of claims 1 to 5, further comprising an adjuvant.

7. The composition according to claim 6, wherein the adjuvant is a balanced Th1/Th2 adjuvant.

8. The composition according to claim 7, wherein the adjuvant is DC-Chol.

9. A composition comprising, in a combined amount effective to generate a significant therapeutic anti-Helicobacter immune response upon administration to an animal having a Helicobacter infection:
(a) the Helicobacter 76K protein or a peptide from said Helicobacter 76K protein; or a nucleic acid that encodes said Helicobacter 76K protein or peptide; or an antibody, or antigen binding fragment thereof that binds to said Helicobacter 76K protein or peptide;
(b) the Helicobacter catalase or a peptide from said Helicobacter catalase; or a nucleic acid that encodes said Helicobacter catalase or peptide; or an antibody, or antigen binding fragment thereof that binds to said Helicobacter catalase or peptide; and
(c) the Helicobacter 525 protease or a peptide from said Helicobacter 525 protease; or a nucleic acid that encodes said Helicobacter 525 protease or peptide; or an antibody, or antigen binding fragment thereof, that binds to said Helicobacter 525 protease or peptide.

10. The composition according to claim 9, further comprising a fourth immunogenic Helicobacter component which is selected from the group consisting of:
(a) the Helicobacter urease or a peptide from said Helicobacter urease; or a nucleic acid that encodes said Helicobacter urease or peptide; or an antibody, or antigen binding fragment thereof that binds to said Helicobacter urease or peptide; and
(b) the Helicobacter AlpA protein or a peptide from said Helicobacter AlpA protein; or a nucleic acid that encodes said Helicobacter AlpA protein or peptide; or an antibody, or antigen binding fragment thereof, that binds to said Helicobacter AlpA protein or peptide.

11. A composition comprising at least a first and second immunogenic Helicobacter component in a combined amount effective to generate a significant therapeutic anti-Helicobacter immune response upon administration to an animal having a Helicobacter infection, wherein:
(a) said at least first immunogenic Helicobacter component is the Helicobacter AlpA protein or a peptide from said Helicobacter AlpA protein; or a nucleic acid that encodes said Helicobacter AlpA protein or peptide; or an antibody, or antigen binding fragment thereof, that binds to said Helicobacter AlpA protein or peptide; and
(b) said at least second immunogenic Helicobacter component is (i) the Helicobacter 76K protein or a peptide from said Helicobacter 76K protein; or a nucleic acid that encodes said Helicobacter 76K protein or peptide; or an antibody, or antigen binding fragment thereof, that binds to said Helicobacter 76K protein or peptide or (ii) Helicobacter catalase or a peptide from said Helicobacter catalase; or a nucleic acid that encodes said Helicobacter catalase or peptide; or an antibody, or antigen binding fragment thereof that binds to said Helicobacter catalase or peptide.

12. The composition according to any one of claims 9 to 11, wherein the 76K protein is BabB.

13. The composition according to any one of claims 9 to 12, further comprising an adjuvant.

14. The composition according to claim 13, wherein the adjuvant is a balanced Th1/Th2 adjuvant.

15. The composition according to claim 14, wherein the adjuvant is DC-Chol.

16. A vaccine comprising the composition according to any one of claims 1 to 15, in a pharmaceutically acceptable excipient.

17. The use of a composition according to any one of claims 1 to 8, in the preparation of a vaccine for protecting an animal against Helicobacter infection.

18. The use of a composition according to any one of claims 9 to 15, in the preparation of a vaccine for treating Helicobacter infection in an animal.
